(19) United States
(12) Patent Application Publication
(10) Pub. No.: US 2018/0133687 A1
(43) Pub. Date: May 17, 2018

(54) Amphiphilic Film Structure Having Hydrophilic and Hydrophobic Properties

(71) Applicant: National University of Kaohsiung, Kaoshsiung (TW)

(72) Inventors: Yi-Chang Chung, Kaoshsiung (TW); Kai-Ming WU, Kaoshsiung (TW)

(21) Appl. No.: 15/462,029

(22) Filed: Mar. 17, 2017

(30) Foreign Application Priority Data
Nov. 14, 2016 (TW) 105137137

(51) Int. Cl. B01J 20/28; B32B 7/02; B32B 7/10; B32B 27/08; B32B 3/26; B32B 27/30; B01J 20/26; A61L 15/52; A61L 15/60; A61L 15/22; A61L 15/24

(52) U.S. Cl. CPC B01J 20/28014; B32B 2419/00; B32B 7/10; B32B 27/08; B32B 3/266; B32B 3/263; B32B 27/308; B01J 20/261; B01J 20/267; A61L 15/52; A61L 15/60; A61L 15/225; A61L 15/24; B32B 2250/24; B32B 2307/728; B32B 2307/73; B32B 2535/00; B32B 2457/00; B32B 7/02

(57) ABSTRACT

An amphiphilic film structure includes an amphiphilic film, a hydrophobic material layer formed as a first material layer and a hydrophilic material layer formed as a second material layer. The hydrophobic material layer is made of at least one hydrophobic monomer material and the hydrophilic material layer is made of at least one hydrophilic monomer material. The hydrophilic material layer is attached to a predetermined surface for absorbing moisture thereof which can be diffused via the hydrophobic material layer.

### BACKGROUND OF THE INVENTION

### 1. Field of the Invention

The present invention relates to an amphiphilic film structure having hydrophilic and hydrophobic properties. Particularly, the present invention relates to the amphiphilic film structure having microstructure to provide hydrophilic and hydrophobic properties.

### 2. Description of the Related Art

U.S. Patent Application Publication No. 2012/0121858, entitled “Hydrophobic film, patterned film having hydrophobic and hydrophilic regions, and method for producing the same,” discloses a conventional superhydrophobic film. The superhydrophobic film is composed of a polymer which have surface microstructures. A surface of the superhydrophobic film includes hydrophobic regions and hydrophilic regions.

PCT Patent Publication No. WO-1991008724, entitled “An improved wound bandage,” discloses a conventional wound bandage. The wound bandage is made of a hydrophobic material for attaching to a wound surface. The hydrophobic material may be a microporous material.

U.S. Patent Application Publication No. 2007/0102348, entitled “Method of producing a porous membrane and waterproof, highly breathable fabric including the membrane,” discloses a conventional breathable fabric which is porous and waterproof. The breathable fabric is made of hydrophobic plastic (such as PVDF).

However, there is a need of improving a conventional dry adhesive tape which cannot adhere to a wet surface or a rough (scraggy) surface. The above-mentioned patent publications are incorporated herein by reference for purposes including, but not limited to, indicating the background of the present invention and illustrating the situation of the art.

As is described in greater detail below, the present invention provides an amphiphilic film structure which is formed with a hydrophilic material layer and a hydrophobic material layer. The hydrophilic material layer is attached to a predetermined surface for absorbing moisture which can be diffused via the hydrophobic material layer in such a way as to mitigate and overcome the above problem.

### SUMMARY OF THE INVENTION

The primary objective of this invention is to provide an amphiphilic film structure which is formed with a hydrophilic material layer and a hydrophobic material layer. The hydrophilic material layer is attached to a predetermined surface for absorbing moisture which can be diffused via the hydrophobic material layer. Advantageously, the amphiphilic film structure of the present invention is successful in directly adhering to a wet surface or a rough surface.

The amphiphilic film structure in accordance with an aspect of the present invention includes:

an amphiphilic film including a first material layer and a second material layer combined therewith;

a hydrophobic material layer formed in a first surface of the amphiphilic film, with the hydrophobic material layer made of at least one hydrophobic monomer material; and

a hydrophilic material layer formed in a second opposite surface of the amphiphilic film, with the hydrophilic material layer made of at least one hydrophilic monomer material;

wherein the hydrophilic material layer is attached to a predetermined surface for absorbing moisture thereof which can be diffused via the hydrophobic material layer.

The amphiphilic film structure in accordance with an aspect of the present invention includes:

an amphiphilic film including a first material layer, a second material layer and a third material layer, with the third material layer including a first side surface on which to combine the first material layer and a second side surface on which to combine the second material layer;

a hydrophobic material layer formed in a first surface of the amphiphilic film, with the hydrophobic material layer made of at least one hydrophobic monomer material;

a hydrophilic material layer formed in a second opposite surface of the amphiphilic film, with the hydrophilic material layer made of at least one hydrophilic monomer material; and

a cross-linking layer formed in the third material layer;

wherein the hydrophilic material layer is attached to a predetermined surface for absorbing moisture thereof which can be diffused via the hydrophobic material layer.

In a separate aspect of the present invention, the hydrophilic monomer material is provided to form or polymerize a homopolymer or a copolymer having a hydrophilic radical group.

In a further separate aspect of the present invention, the hydrophilic radical group includes a primary amine, a secondary amine, a tertiary amine, a quaternary ammonium salt, a hydroxyl, a carboxyl, a sulfonic acid group, a phosphoric acid group, an amide group or an ionic hydrophilic group.

In yet a further separate aspect of the present invention, the hydrophobic monomer material is provided to form or polymerize a homopolymer or a copolymer having a hydrophobic radical group.

In yet a further separate aspect of the present invention, the hydrophobic radical group includes a predetermined long carbon chain, a predetermined cyclic carbon chain, a predetermined fluorocarbon chain or a predetermined benzene.

In yet a further separate aspect of the present invention, the hydrophobic material layer includes a plurality of first ventilation holes arranged thereon.

In yet a further separate aspect of the present invention, the hydrophilic material layer includes a plurality of second ventilation holes arranged thereon.

In yet a further separate aspect of the present invention, the first ventilation hole is aligned with the second ventilation hole.

In yet a further separate aspect of the present invention, the hydrophobic material layer includes a plurality of first microstructures.

In yet a further separate aspect of the present invention, the hydrophobic material layer includes a plurality of second microstructures.

Further scope of the applicability of the present invention will become apparent from the detailed description given hereinafter. However, it should be understood that the detailed description and specific examples, while indicating preferred embodiments of the invention, are given by way of illustration only, since various will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIG. 1 is a side view of an amphiphilic film structure in accordance with a first preferred embodiment of the present invention.

FIG. 1A is an image view of an included angle between a water drop edge and a hydrophobic flat surface of the amphiphilic film structure in accordance with the first preferred embodiment of the present invention.

FIG. 1B is an image view of an included angle between a water drop edge and a hydrophilic flat surface of the amphiphilic film structure in accordance with the first preferred embodiment of the present invention.

FIG. 2 is a side view of an amphiphilic film structure in accordance with a second preferred embodiment of the present invention.

FIG. 3 is a side view of an amphiphilic film structure in accordance with a third preferred embodiment of the present invention.

FIG. 4 is a side view of an amphiphilic film structure in accordance with a fourth preferred embodiment of the present invention.

FIG. 5 is a side view of an amphiphilic film structure in accordance with a fifth preferred embodiment of the present invention.

FIG. 6 is a side view of an amphiphilic film structure in accordance with a sixth preferred embodiment of the present invention.

FIG. 7 is a side view of an amphiphilic film structure in accordance with a seventh preferred embodiment of the present invention.

FIG. 7A is an image view of an included angle between a water drop edge and a hydrophobic microstructure surface of the amphiphilic film structure in accordance with the seventh preferred embodiment of the present invention.

FIG. 7B is an image view of an included angle between a water drop edge and a hydrophilic microstructure surface of the amphiphilic film structure in accordance with the seventh preferred embodiment of the present invention.

FIG. 8 is a side view of an amphiphilic film structure in accordance with an eighth preferred embodiment of the present invention.

FIGS. 9(a) to 9(g) are chemical structure views of a series of hydrophilic radical groups of amine monomer materials applied in the amphiphilic film structure in accordance with a preferred embodiment of the present invention.

FIG. 10 is a chemical structure view of a radical group of a hydrophobic monomer material applied in the amphiphilic film structure in accordance with a preferred embodiment of the present invention.

### DETAILED DESCRIPTION OF THE INVENTION

It is noted that an amphiphilic film structure in accordance with the present invention can be applicable for sticking various products, including optical glass, LCD panels, solar cells, semiconductor elements, silicon wafers, 3C products, metal or steel-protective films of building materials, plastic products and spare parts or other protection-required objects, for example, which are not limitative of the present invention.

Additionally, the amphiphilic film structure of the present invention is suitable for various medical products, medical aid products (i.e. bandage or wound plaster, medical aesthetic products (i.e. wrinkle patch), medical rehabilitation products (i.e. surgery patch), medicine products, sport products (i.e. athletic protective tape) or other related products, for example, which are not limitative of the present invention.

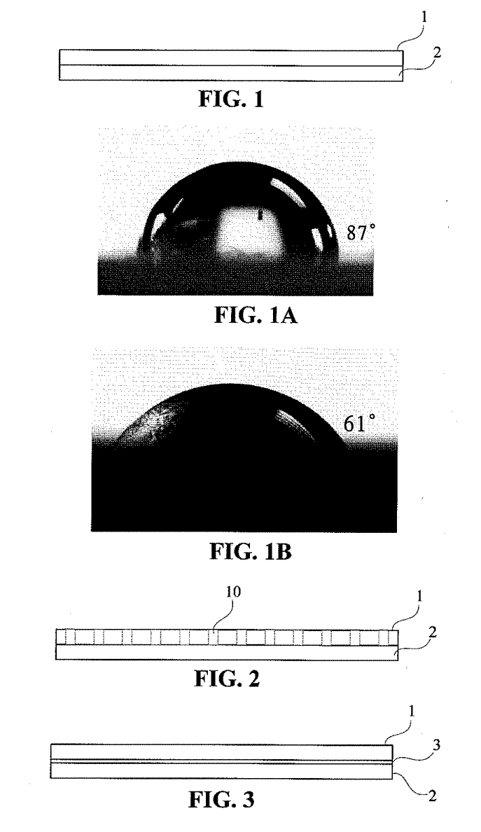

FIG. 1 shows a side view of an amphiphilic film structure in accordance with a first preferred embodiment of the present invention. Referring now to FIG. 1, the amphiphilic film structure of the first preferred embodiment of the present invention includes an amphiphilic (patch) body, at least one hydrophobic material layer 1 and at least one hydrophilic material layer 2. In a preferred embodiment, the amphiphilic film structure includes a plurality of hydrophobic material layers 1 and a plurality of hydrophilic material layers 2, one hydrophobic material layer 1 and a plurality of hydrophilic material layers 2, or a plurality of hydrophobic material layers 1 and one hydrophilic material layer 2.

With continued reference to FIG. 1, the amphiphilic body includes a first material layer and a second material layer combined therewith. The hydrophobic material layer 1 is formed in a first surface of the amphiphilic body and is made of at least one hydrophobic monomer material. Correspondingly, the hydrophilic material layer 2 is formed in a second opposite surface of the amphiphilic body and is made of at least one hydrophilic monomer material. In a preferred embodiment, a first adjacent surface layer of the first material layer is integrated into a second adjacent surface layer of the second material layer in production.

Still referring to FIG. 1, the hydrophilic material layer 2 is attached to a predetermined surface (i.e. wound surface) for absorbing moisture (i.e. wound fluid) thereof. Meanwhile, the absorbed or oozed moisture or fluid can be continuously diffused via the hydrophobic material layer 1 to exterior. Advantageously, this results in enhancing the reliability of viscosity of the amphiphilic body.

In a preferred embodiment, the hydrophilic monomer material is provided to form or polymerize a homopolymer or a copolymer having a hydrophilic radical group. The hydrophilic radical group includes a primary amine, a secondary amine, a tertiary amine, a quaternary ammonium salt, a hydroxyl, a carboxyl, a sulfonic acid group, a phosphoric acid group, an amide group or an ionic hydrophilic group. In a preferred embodiment, the hydrophobic monomer material is provided to form or polymerize a homopolymer or a copolymer having a hydrophobic radical group. The hydrophobic radical group includes a predetermined long carbon chain, a predetermined cyclic carbon chain, a predetermined fluorocarbon chain or a predetermined benzene.

FIG. 1A shows an image view of an included angle between a water drop edge and a hydrophobic flat surface of the amphiphilic film structure in accordance with the first preferred embodiment of the present invention. Referring now to FIGS. 1 and 1A, a water drop is dripped down on the hydrophobic material layer 1. An included angle between a water drop edge and a hydrophobic flat surface reaches up to 87 degrees.

FIG. 1B shows an image view of an included angle between a water drop edge and a hydrophilic flat surface of the amphiphilic film structure in accordance with the first preferred embodiment of the present invention. Referring now to FIGS. 1 and 1B, another water drop is dripped down on the hydrophilic material layer 2. In the beginning, an included angle between a water drop edge and a hydrophilic flat surface can still reach up to 61 degrees. After a predetermined time, the hydrophilic material layer 2 completely absorbs the water drop and the surface expands.

FIG. 2 shows a side view of an amphiphilic film structure in accordance with a second preferred embodiment of the present invention. Referring to FIG. 2, in comparison with the first embodiment, the hydrophobic material layer 1 of the second preferred embodiment includes a plurality of first ventilation holes 10 which are equi-spaced on the surface of the hydrophobic material layer 1. The first ventilation holes 10 can accelerate the diffusion of moisture to exterior when the amphiphilic body is attached to a wound surface.

FIG. 3 shows a side view of an amphiphilic film structure in accordance with a third preferred embodiment of the present invention. Referring to FIG. 2, the amphiphilic film structure of the third preferred embodiment of the present invention includes an amphiphilic (patch) body, at least one hydrophobic material layer 1, at least one hydrophilic material layer 2 and a cross-linking layer 3.

With continued reference to FIG. 3, the amphiphilic body includes a first material layer, a second material layer and a third material layer which are combined together. The cross-linking layer 3 has a first side surface on which to combine the hydrophobic material layer 1 and a second side surface on which to combine the hydrophilic material layer 2. The hydrophobic material layer 1 is made of at least one hydrophobic monomer material while the hydrophilic material layer 2 is made of at least one hydrophilic monomer material. The cross-linking layer 3 is made of a photo cross-linking material, a thermal cross-linking material, a double functional group material or a multi functional group material.

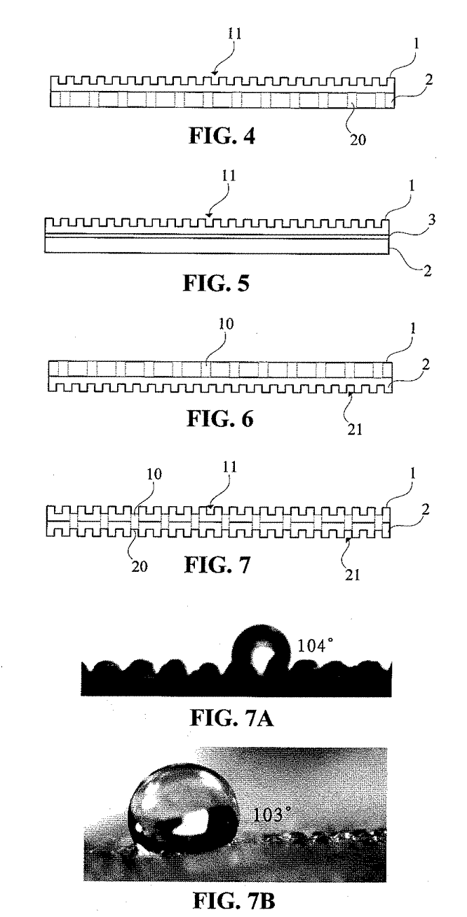

FIG. 4 shows a side view of an amphiphilic film structure in accordance with a fourth preferred embodiment of the present invention. Referring to FIG. 4, in comparison with the first embodiment, the hydrophilic material layer 2 of the fourth preferred embodiment includes a plurality of second ventilation holes 20 which are equi-spaced on the surface of the hydrophilic material layer 2. The second ventilation holes 20 can accelerate the diffusion of moisture to exterior when the amphiphilic body is attached to a wound surface. In a preferred embodiment, in addition to the second ventilation holes 20, the hydrophobic material layer 1 further includes a plurality of first ventilation holes 10 which may be in perfect alignment with the second ventilation holes 20.

FIG. 5 shows a side view of an amphiphilic film structure in accordance with a fifth preferred embodiment of the present invention. Referring to FIG. 5, in comparison with the third embodiment, the hydrophobic material layer 1 of the fifth preferred embodiment includes a plurality of first microstructures 11 which are formed on a surface of the hydrophobic material layer 1.

FIG. 6 shows a side view of an amphiphilic film structure in accordance with a sixth preferred embodiment of the present invention. Referring to FIG. 5, in comparison with the first embodiment, the hydrophilic material layer 2 of the sixth preferred embodiment includes a plurality of second microstructures 21 which are formed on a surface of the hydrophilic material layer 2.

FIG. 7 shows a side view of an amphiphilic film structure in accordance with a seventh preferred embodiment of the present invention. Referring to FIG. 7, in comparison with the first embodiment, the hydrophobic material layer 1 of the seventh preferred embodiment includes a plurality of first ventilation holes 10 and a plurality of first microstructures 11. Correspondingly, the hydrophilic material layer 2 of the seventh preferred embodiment includes a plurality of second ventilation holes 20 and a plurality of second microstructures 21.

FIG. 7A shows an image view of an included angle between a water drop edge and a hydrophobic microstructure surface of the amphiphilic film structure in accordance with the seventh preferred embodiment of the present invention. Referring now to FIGS. 7 and 7A, a water drop is dripped down on the first microstructures 11 of the hydrophobic material layer 1. An included angle between a water drop edge and a hydrophobic surface reaches up to 104 degrees.

FIG. 7B shows an image view of an included angle between a water drop edge and a hydrophilic microstructure surface of the amphiphilic film structure in accordance with the seventh preferred embodiment of the present invention. Referring now to FIGS. 7 and 7B, another water drop is dripped down on the second microstructures 21 of the hydrophilic material layer 2. In the beginning, an included angle between a water drop edge and a hydrophilic surface can still reach up to 103 degrees. After a predetermined time, the hydrophilic material layer 2 completely absorbs the water drop and the surface expands.

FIG. 8 shows a side view of an amphiphilic film structure in accordance with an eighth preferred embodiment of the present invention. Referring to FIG. 8, in comparison with the first embodiment, the hydrophobic material layer 1 of the eighth preferred embodiment includes a plurality of first microstructures 11 arranged on a surface opposite to the cross-linking layer 3. Correspondingly, the hydrophilic material layer 2 of the eighth preferred embodiment includes a plurality of second microstructures 21 arranged on a surface opposite to the cross-linking layer 3.

FIGS. 9(a) to 9(g) show chemical structure views of a series of hydrophilic radical groups of amine monomer materials applied in the amphiphilic film structure in accordance with a preferred embodiment of the present invention. Referring to FIGS. 9(a) to 9(g), seven types of amine monomer materials are exemplified for manufacturing the hydrophilic material layer 2 and combinations of the amine monomer materials are also suitable for manufacturing the hydrophilic material layer 2.

FIG. 10 shows a chemical structure view of a radical group of a hydrophobic monomer material applied in the amphiphilic film structure in accordance with a preferred embodiment of the present invention. Turning now to FIG. 10, a hydrophobic monomer material is selected from an ether acrylic monomer having a long carbon chain suitable for manufacturing the hydrophobic material layer 1. A carbon number of the carbon chain of the hydrophobic monomer material is preferably 1 to 12. Furthermore, the hydrophobic monomer material is selected from dipropylene glycol methyl ether diacrylate, propylene glycol methyl ether acrylate and isomers thereof.

Although the invention has been described in detail with reference to its presently preferred embodiment, it will be understood by one of ordinary skill in the art that various modifications can be made without departing from the spirit and the scope of the invention, as set forth in the appended claims.

What is claimed is:

1. An amphiphilic film structure comprising:
an amphiphilic film including a first material layer and a second material layer combined therewith;
a hydrophobic material layer formed in a first surface of the amphiphilic film, with the hydrophobic material layer made of at least one hydrophobic monomer material; and
a hydrophilic material layer formed in a second opposite surface of the amphiphilic film, with the hydrophilic material layer made of at least one hydrophilic monomer material;
wherein the hydrophilic material layer is attached to a predetermined surface for absorbing moisture thereof which can be diffused via the hydrophobic material layer.

2. The amphiphilic film structure as defined in claim 1, wherein the hydrophilic monomer material is provided to form or polymerize a homopolymer or a copolymer having a hydrophilic radical group.

3. The amphiphilic film structure as defined in claim 2, wherein the hydrophilic radical group includes a primary amine, a secondary amine, a tertiary amine, a quaternary ammonium salt, a hydroxyl, a carboxyl, a sulfonic acid group, a phosphoric acid group, an amide group or an ionic hydrophilic group.

4. The amphiphilic film structure as defined in claim 1, wherein the hydrophobic monomer material is provided to form or polymerize a homopolymer or a copolymer having a hydrophobic radical group.

5. The amphiphilic film structure as defined in claim 4, wherein the hydrophobic radical group includes a predetermined long carbon chain, a predetermined cyclic carbon chain, a predetermined fluorocarbon chain or a predetermined benzene.

6. The amphiphilic film structure as defined in claim 1, wherein the hydrophobic material layer includes a plurality of first ventilation holes arranged thereon.

7. The amphiphilic film structure as defined in claim 1, wherein the hydrophilic material layer includes a plurality of second ventilation holes arranged thereon.

8. The amphiphilic film structure as defined in claim 1, wherein the hydrophobic material layer includes a plurality of first ventilation holes and the hydrophilic material layer includes a plurality of second ventilation holes, with the first ventilation hole aligned with the second ventilation hole.

9. The amphiphilic film structure as defined in claim 1, wherein the hydrophobic material layer includes a plurality of first microstructures.

10. The amphiphilic film structure as defined in claim 1, wherein the hydrophobic material layer includes a plurality of second microstructures.

11. An amphiphilic film structure comprising:
an amphiphilic film including a first material layer, a second material layer and a third material layer, with the third material layer including a first side surface on which to combine the first material layer and a second side surface on which to combine the second material layer,
a hydrophobic material layer formed in a first surface of the amphiphilic film, with the hydrophobic material layer made of at least one hydrophobic monomer material;
a hydrophilic material layer formed in a second opposite surface of the amphiphilic film, with the hydrophilic material layer made of at least one hydrophilic monomer material; and
a cross-linking layer formed in the third material layer;
wherein the hydrophilic material layer is attached to a predetermined surface for absorbing moisture thereof which can be diffused via the hydrophobic material layer.

12. The amphiphilic film structure as defined in claim 11, wherein, the hydrophilic monomer material is provided to form or polymerize a homopolymer or a copolymer having a hydrophilic radical group.

13. The amphiphilic film structure as defined in claim 12, wherein the hydrophilic radical group includes a primary amine, a secondary amine, a tertiary amine, a quaternary ammonium salt, a hydroxyl, a carboxyl, a sulfonic acid group, a phosphoric acid group, an amide group or an ionic hydrophilic group.

14. The amphiphilic film structure as defined in claim 11, wherein the hydrophobic monomer material is provided to form or polymerize a homopolymer or a copolymer having a hydrophobic radical group.

15. The amphiphilic film structure as defined in claim 14, wherein the hydrophobic radical group includes a predetermined carbon chain, a predetermined cyclic carbon chain, a predetermined fluorocarbon chain or a predetermined benzene.

16. The amphiphilic film structure as defined in claim 11, wherein the hydrophobic material layer includes a plurality of first ventilation holes arranged thereon.

17. The amphiphilic film structure as defined in claim 11, wherein the hydrophilic material layer includes a plurality of second ventilation holes arranged thereon.

18. The amphiphilic film structure as defined in claim 11, wherein the hydrophobic material layer includes a plurality of first ventilation holes and the hydrophilic material layer includes a plurality of second ventilation holes, with the first ventilation hole aligned with the second ventilation hole.

19. The amphiphilic film structure as defined in claim 11, wherein the hydrophobic material layer or the hydrophobic material layer includes a plurality of microstructures.

20. The amphiphilic film structure as defined in claim 11, wherein the cross-linking layer is made of a photo cross-linking material, a thermal cross-linking material, a double functional group material or a multi functional group material.
